(19) United States
(12) Patent Application Publication
(10) Pub. No.: US 2012/0283805 A1
(43) Pub. Date: Nov. 8, 2012

(54) NECK WARMER WITH INCREASED ERGONOMICS AND EFFICIENCY, PARTICULARLY FOR CERVICAL THERMOTERAPY

(76) Inventors: Roberto Ragonesi, Firenze (IT); Francesco Barabino, Montespertoli (IT)

(21) Appl. No.: 13/099,245

(22) Filed: May 2, 2011

(51) Int. Cl. A61F 7/08

(52) U.S. Cl. 607/109

(57) ABSTRACT

A neck warmer with increased ergonomics and efficiency, particularly for cervical thermotherapy, comprising a band made of fabric having, at a central portion, at least one pocket for accommodating a heating element, wherein the fabric band is associated substantially along its entire length with at least one substantially C-shaped preformed stiffening lamina, for wrapping it around a user's neck to be subjected to heat treatment, the stiffening lamina can be shaped manually to adapt its shape to the shape of the neck to be subjected to heat treatment, the portion of the fabric band in contact with the neck to be subjected to heat treatment can be provided with a heat-conducting strip for the diffusion of the heat generated by the heating element along the entire length of the fabric band and thus along the entire part of the neck to be subjected to heat treatment in contact with the fabric band, moreover, there can be a layer of shape memory material defined on the inner side of the stiffening lamina to increase the comfort and fit of the neck warmer, the access of the pocket can be defined on the outer side of the fabric band to prevent direct contact between the skin of the user and the heating element.

The present invention relates to a neck warmer with increased ergonomics and efficiency, particularly for cervical thermotherapy.

### BACKGROUND OF THE INVENTION

As is known, stiff neck conditions, shoulder rheumatisms and/or pain in the cervical vertebrae caused by atmospheric conditions or by increasingly sedentary life are a problem that affects many people, particularly in winter periods or during damp and colder days.

To reduce these symptoms, it is known to perform rehabilitation therapies, such as for example localized massages performed by specialized physiotherapists or thermotherapy applications by using warming pads.

In both cases, however, high intervention costs are encountered because it is necessary to perform several prolonged therapies due to the persistence of the symptom.

In both cases, moreover, it is necessary to resort to treatment facilities, with consequent difficulties in getting appointments in a short time and with constant frequency, and this worsens the pain and therefore the physical discomfort, and has a negative effect on the private life and working life of the patient.

Finally, these treatments require the user to physically go to dedicated facilities which require considerable amounts of time for transfers and treatments; time which is taken from the leisure time and/or work time of the patient and therefore has a cost.

To obviate the problems described above, soft collar devices for cervical thermotherapies have been devised.

More precisely, these collar devices consist of a padded fabric band which is provided with a pocket adapted to accommodate a heating element.

A stiffening element is also provided, which passes through the fabric band along its longitudinal length in order to give it a substantially C-like shape, so as to be able to wrap around the neck to be subjected to heat treatment.

In order to make the collar device wearable on any neck size, a collar device is disclosed in EP 1264584 in which the stiffening element can be made of a material that is plastically deformable at room temperature directly by the user, so as to be able to change its curvature and shape it manually according to the requirements.

These conventional collar devices are not devoid of drawbacks, which include the fact that since the pocket that accommodates the heating element is defined entirely on the inner side of the fabric band, if the heating element exits accidentally from the pocket it might come into contact with the user's skin, consequently scalding it. The heating element in fact works with an operating temperature close to 80° C.

Another drawback of conventional collar devices is that they are unable to provide uniform heating along the entire extension of the fabric band. Since there is a single pocket that accommodates the heating element, effective heat diffusion occurs only at the portion provided with the pocket, which is generally the central portion of the band.

A further drawback of conventional collar devices is that they do not follow perfectly the shape of the neck of the user, despite the possibility to shape the stiffening element at will.

### SUMMARY OF THE INVENTION

The aim of the present invention is to overcome and solve, respectively, the limitations and drawbacks of the background art, by providing a neck warmer that improves heat distribution around the neck of the user although the source that generates the heat has a very small extension with respect to the circumference of the neck.

Another object of the invention is to provide a neck warmer that allows more effective adaptation to the shape of the neck of any user.

A further object of the invention is to provide a neck warmer that prevents accidental scalding of the user due to direct contact between the heat source and the neck.

Another object of the present invention is to provide a device that has a low production cost and is structurally simple.

This aim, as well as these and other objects which will become better apparent hereinafter, are achieved by a neck warmer with increased ergonomics and efficiency, particularly for cervical thermotherapy, comprising a band made of fabric which has, at a central portion, at least one pocket for accommodating a heating element, the fabric band being associated substantially along its entire length with a substantially C-shaped preformed stiffening lamina, which is adapted to fit the fabric band on a neck of a user and can be shaped manually to adapt the shape of the lamina to the shape of the neck to be subjected to heat treatment, wherein the inner side of the fabric band in contact with the neck to be subjected to heat treatment is provided with a heat-conducting strip for the diffusion of the heat generated by said heating element along the entire length of the fabric band.

This aim and these and other objects are also achieved by a neck warmer with increased ergonomics and efficiency, particularly for cervical thermotherapy, comprising a band made of fabric which has, at a central portion, at least one pocket for accommodating a heating element, the fabric band being associated substantially along its entire length with a substantially C-shaped preformed stiffening lamina, which is adapted to fit the fabric band on a neck of a user and can be shaped manually to adapt the shape of the lamina to the shape of the neck to be subjected to heat treatment, wherein there is a layer of shape memory material which is fixed on the inner side of the stiffening lamina to increase the comfort and fit of the neck warmer.

This aim and these and other objects are also achieved by a neck warmer with increased ergonomics and efficiency, particularly for cervical thermotherapy, comprising a band made of fabric which has, at a central portion, at least one pocket for accommodating a heating element, the fabric band being associated substantially along its entire length with a substantially C-shaped preformed stiffening lamina, which is adapted to fit the fabric band to a neck of a user and can be shaped manually to adapt the shape of the lamina to the shape of the neck to be subjected to heat treatment, wherein the access of said at least one pocket is defined on the outer side of the fabric band in order to prevent accidental direct contact between the skin of the user and the heating element.

### BRIEF DESCRIPTION OF THE DRAWINGS

Further characteristics and advantages will become better apparent from the description of preferred but not exclusive embodiments of a neck warmer with increased ergonomics and efficiency, particularly for cervical thermotherapy, illustrated by way of non-limiting example in the accompanying drawings, wherein:

FIG. 1 is a front perspective view of a first embodiment of a neck warmer with increased ergonomics and efficiency, particularly for cervical thermotherapy, according to the invention;

FIG. 2 is a rear perspective view of the neck warmer shown in FIG. 1;

FIG. 3 is a perspective view of the stiffening lamina of the neck warmer shown in FIG. 1;

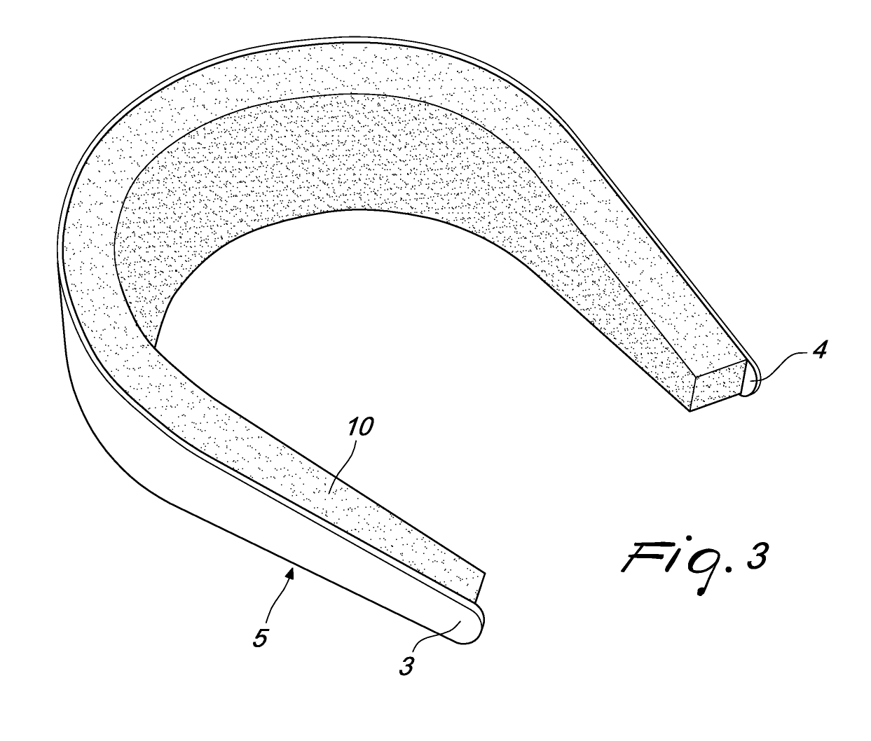

FIG. 4 is a front perspective view of a variation of the neck warmer shown in FIG. 1.

### DESCRIPTION OF THE PREFERRED EMBODIMENTS

With reference to the figures, the neck warmer with increased ergonomics and efficiency, particularly for cervical thermotherapy, generally designated by the reference numeral 1, comprises a fabric band 2 which is shaped so as to be adapted to wrap around the neck to be subjected to heat treatment of a user and is associated with at least one substantially C-shaped preformed stiffening lamina 5.

The fabric of which the fabric band 2 is made can be made of different materials, such as for example synthetic material known as pile or natural material, such as for example wool or cotton or similar materials.

The fabric band 2 is provided, at a central portion, with at least one pocket 6 for accommodating a heating element 7, which is of the removable type and can be constituted by a sachet or capsule of a known type which is adapted to produce an exothermic reaction upon contact with air.

Advantageously, the pocket 6 is preferably formed at the region of the nape of the neck to be subjected to heat treatment and the access 8 of the pocket 6 is defined on the outer side 2a of the fabric band 2 to prevent direct contact between the skin of the user and the heating element 7 in case of accidental escape and incorrect placements thereof.

More precisely, the inside of the pocket 6 is defined on the inner side 2b of the fabric band 2 so as to position the heating element 7 between the stiffening lamina 5 and the neck of the user to be subjected to heat treatment.

As an alternative, the pocket 6 can be obtained directly on the fabric band 2, forming thereon an opening which is for example slit-shaped and arranging thereat a containment pouch or pocket for the heating element 7, which is then arranged between the stiffening lamina 5 and the layer of the fabric band 2 that makes direct contact with the neck of the user.

The pocket 6 can be closed optionally by means of a zip fastener or other known means, such as for example press-studs or hook and loop fasteners such as Velcro®.

As already mentioned, the fabric band 2 is associated with a stiffening lamina 5.

More precisely, this association is allowed by a tunnel-shaped cavity 9 that passes longitudinally through the entire fabric band 2 and in which the stiffening lamina 5 is inserted, thus being concealed from sight.

Advantageously, the stiffening lamina 5, which is per se substantially C-shaped, can be shaped manually by the user at room temperature to adapt its shape to the shape of the neck to be subjected to heat treatment.

The stiffening lamina 5 is made of plastic material, metallic material or similar material so as to be shaped to give it position memory, i.e., retain the shape imposed manually by the user by moving its ends closer or further apart; for example, it can consist of a styrene/butadiene copolymer.

This means that the material of which the stiffening lamina 5 is made can allow the user to impart thereto a shape selected so as to make the fabric band 5 adhere more or less precisely to the neck to be subjected to heat treatment of each individual user.

Geometrically, the stiffening lamina 5 has a height that is approximately equal to the height of the tunnel-shaped cavity 9 in which it is inserted and has rounded ends 3 and 4.

As regards the use, it is sufficient for the user to grip the ends 3 and 4 of the stiffening lamina 5 that are contained within the fabric band 2. To allow the neck warmer 1 to adhere more precisely to the neck to be subjected to heat treatment, it is sufficient to push forcefully the ends 3 and 4 toward each other, cross them and keep them in this crossed position for a few seconds.

On the other hand, to make the neck warmer 1 adhere less tightly to the neck to be subjected to heat treatment, the user, after gripping the ends 3 and 4 of the stiffening lamina 5, simply has to widen them forcefully, moving them mutually apart.

The length of the stiffening lamina 5 is such as to not wrap completely around the user's neck to be subjected to heat treatment, so as to allow the fabric band 2 to adhere to the neck to be subjected to heat treatment in an optimum manner without throttling the neck.

In order to make the neck warmer 1 adhere even more precisely to the neck to be subjected to heat treatment, there can be a layer of shape memory material 10, constituted for example by a foamed material of the memory-foam type, which is fixed to the inner side of the stiffening lamina 5: this makes it possible to further increase the comfort and fit of the neck warmer 1.

The foamed material can be polyurethane, for example of the non-hydrophilic type, of a type known per se.

It is also possible to apply, inside the fabric band 2 and in contact with the stiffening lamina 5, any type of thermally insulating material or thermally insulating fabric that allows a better and more prolonged diffusion of heat along the entire region of the neck and is surrounded by the lamina.

Advantageously, the inner side 2b of the fabric band 2 in contact with the neck to be subjected to heat treatment is provided with a heat-conducting strip 11 for diffusing the heat generated by the heating element 7 along the entire length of the inner side of the fabric band 2.

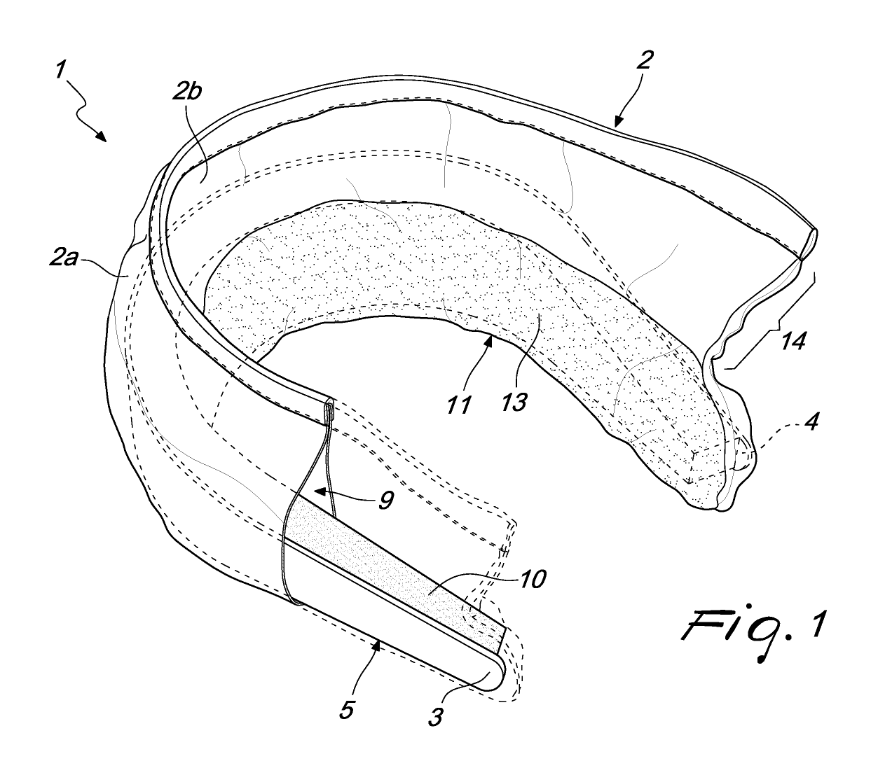

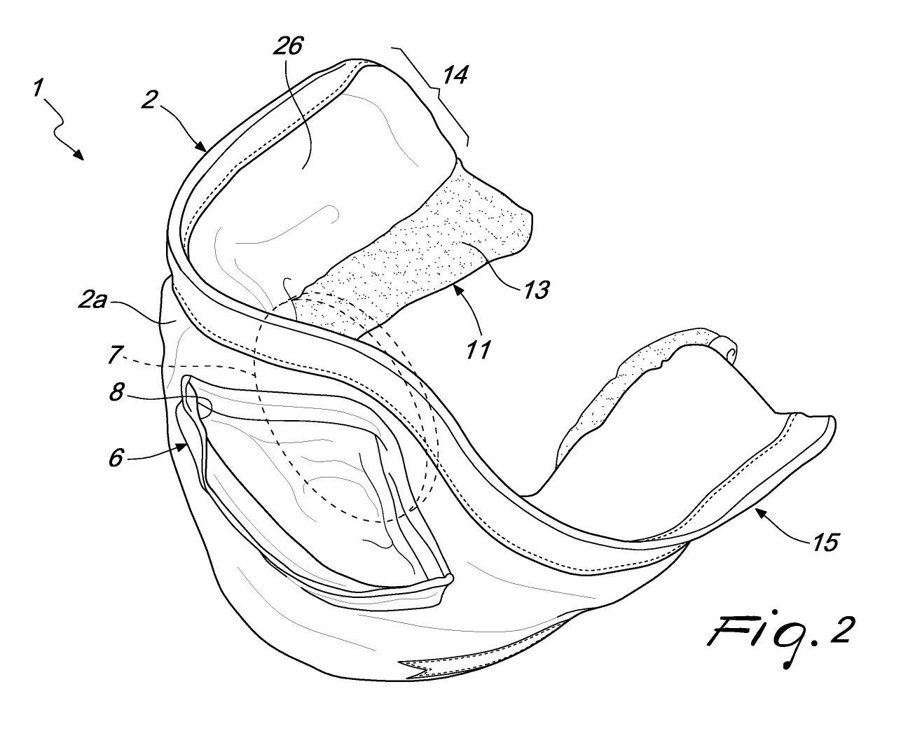

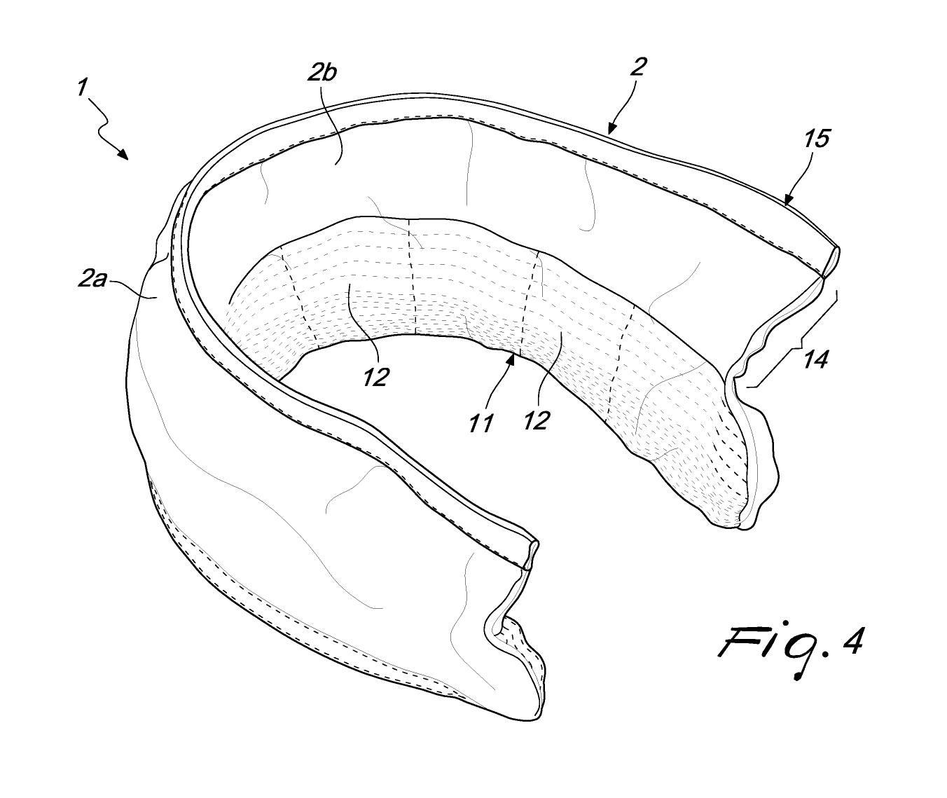

More specifically, as shown in FIGS. 1 and 2, the heat-conducting strip 11 can comprise a layer of heat-conducting material 13 which is deposited on the inner side 2b of the fabric band 2, by means of a wetting or spreading process or, as shown in FIG. 4, it can comprise a weave of filaments 12 made of a heat-conducting material along the inner side 2b of the fabric band 2.

Preferably, the heat-conducting material of which the heat-conducting strip 11 is made, i.e. the layer 13 or each filament of the weave 12, is copper.

Moreover, the fabric band 2 can have, proximate to its ends 3 and 4, adapted engagement or closure means, such as for example press-studs, tapes or bands with hooks and loops known by the trademark Velcro®, which are adapted to keep in mutual contact the front ends 14a and 14b of the fabric band 2.

This additional refinement can be considered optional, since the stiffening lamina 5 thus conceived is such as to ensure by itself a correct adhesion and grip of the fabric band 2 on the neck to be subjected to heat treatment.

In addition or as an alternative to the above, the fabric band 2 can be impregnated with essences of various kinds, such as aloe vera, so as to release beneficial substances during its use.

Finally, the fabric band 2 can have, in an upward region, a raised flap 15 which acts as a collar to protect the neck more effectively against wind and cold air.

If the flap 15 is omitted, the neck warmer 1 has a reduced height and can be applied more discreetly, since in this case the neck warmer 1 proper can be concealed easily by use of a high-neck sweater or any other item of clothing that can be worn by the user.

In order to activate the neck warmer 1 it is sufficient for the user to first open the packet that contains the heating element 7, which can be heated, for example in the form of a sachet or pouch which generates an exothermic reaction upon contact with air, and then insert said sachet into the pocket 6.

The user then grips the fabric band 2 at its front ends, adapting them, as mentioned earlier, to the shape of his neck.

It is then sufficient to apply the neck warmer 1 so that the region provided with the pocket 6 for the heating element 7 rests on the rear portion of the neck and releases the front ends of the fabric band 2, which due to the characteristics of the stiffening lamina 5 adheres to the nape and to the neck, allowing the heat to reach initially the entire neck and then, after a few minutes, also the shoulders and the back of the user.

Thermotherapy thus begins and the beneficial effect of the heat can be perceived immediately.

In practice it has been found that the neck warmer with increased ergonomics and efficiency, particularly for cervical thermotherapy, according to the present invention, achieves the intended aim and objects, allowing the user to have it available immediately when pain begins simply by performing quick operations that do not require specific manual skill or training, adapting perfectly to any neck size and shape, releasing heat uniformly over the entire neck.

The neck warmer according to the present invention can be used anywhere or at any time during the activity of a person, both in daytime and while sleeping at night, without any danger, since there is no electric power supply and the effect ceases simply when the chemical characteristics of the heating element have been depleted.

Another advantage of the neck warmer according to the present invention is that it can be so comfortable that it can be used both as a simple system for getting warm and as an accessory for therapies for the treatment of cervical problems, stiff necks or simple rheumatism.

Another advantage of the neck warmer according to the present invention is that it has low production costs.

The neck warmer with increased ergonomics and efficiency, particularly for cervical thermotherapy, thus conceived is susceptible of numerous modifications and variations, all of which are within the scope of the same inventive concept.

All the details may further be replaced with other technically equivalent elements.

In practice, the materials used, as long as they are compatible with the specific use, as well as the contingent shapes and dimensions, may be any according to requirements.

What is claimed is:

1. A neck warmer with increased ergonomics and efficiency, particularly for cervical thermotherapy, comprising a band made of fabric which has, at a central portion, at least one pocket for accommodating a heating element, said fabric band being associated substantially along its entire length with a substantially C-shaped preformed stiffening lamina, which is adapted to fit said fabric band on a neck of a user and can be shaped manually to adapt the shape of said lamina to the shape of the neck to be subjected to heat treatment, wherein the inner side of said fabric band in contact with the neck to be subjected to heat treatment is provided with a heat-conducting strip for the diffusion of the heat generated by said heating element along the entire length of said fabric band.

2. The neck warmer according to claim 1, wherein said heat-conducting strip comprises a weave of filaments made of a heat-conducting material, said weave of filaments being applied along said inner side of said fabric band in contact with the neck to be subjected to heat treatment.

3. The neck warmer according to claim 1, wherein said heat-conducting strip comprises a layer of heat-conducting material which is deposited on said inner side of said fabric band in contact with the neck to be subjected to heat treatment.

4. The neck warmer according to claim 2, wherein said heat-conducting material is copper.

5. The neck warmer according to claim 3, wherein said heat-conducting material is copper.

6. The neck warmer according to claim 1, wherein said at least one stiffening lamina is made of a styrene/butadiene copolymer.

7. The neck warmer according to claim 1, wherein said fabric band is impregnated with at least one essence.

8. A neck warmer with increased ergonomics and efficiency, particularly for cervical thermotherapy, comprising a band made of fabric which has, at a central portion, at least one pocket for accommodating a heating element, said fabric band being associated substantially along its entire length with a substantially C-shaped preformed stiffening lamina, which is adapted to fit said fabric band on a neck of a user and can be shaped manually to adapt the shape of the lamina to the shape of the neck to be subjected to heat treatment, wherein there is a layer of shape memory material which is fixed to the inner side of said stiffening lamina to increase the comfort and fit of said neck warmer.

9. The neck warmer according to claim 8, wherein said layer of shape memory material is a foamed material.

10. The neck warmer according to claim 8, wherein said at least one stiffening lamina is made of a styrene/butadiene copolymer.

11. The neck warmer according to claim 8, wherein said fabric band is impregnated with at least one essence.

12. A neck warmer with increased ergonomics and efficiency, particularly for cervical thermotherapy, comprising a band made of fabric which has, at a central portion, at least one pocket for accommodating a heating element, said fabric band being associated substantially along its entire length with at least one substantially C-shaped preformed stiffening lamina, which is adapted to fit said fabric band to a neck of a user and can be shaped manually to adapt the shape of said lamina to the shape of the neck to be subjected to heat treatment, wherein the access of said at least one pocket is defined on the outer side of said fabric band in order to prevent accidental direct contact between the skin of the user and said heating element.

13. The neck warmer according to claim 12, wherein the inside of said at least one pocket is defined on an inner side of said fabric band to position said heating element between said stiffening lamina and the user's neck to be subjected to heat treatment.

14. The neck warmer according to claim 12, wherein said at least one stiffening lamina is made of a styrene/butadiene copolymer.

15. The neck warmer according to claim 12, wherein said fabric band is impregnated with at least one essence.
